(19) United States
(12) Patent Application Publication
(10) Pub. No.: US 2008/0132791 A1
(43) Pub. Date: Jun. 5, 2008

(54) SINGLE FRAME - MULTIPLE FREQUENCY COMPOUNDING FOR ULTRASOUND IMAGING

(76) Inventor: Harold M. Hastings, Garden City, NY (US)

Correspondence Address: PROSKAUER ROSE LLP;PATENT DEPARTMENT, 1585 BROADWAY, NEW YORK, NY, 10036-8299, US

(21) Appl. No.: 11/947,462

(22) Filed: Nov. 29, 2007

Related U.S. Application Data
(60) Provisional application No. 60/867,922, filed on Nov. 30, 2006.

(51) Int. Cl. A61B 8/14

(52) U.S. Cl. 600/447

(57) ABSTRACT

When an ultrasound transducer is driven by a signal that contains a relatively wide range of frequencies, the frequency-dependent attenuation characteristics of the subject being imaged can be relied on to simultaneously provide, using only a single pulse per line of the image, (a) a return from the deeper portions of the image that is dominated by lower frequencies and (b) a return from the shallower portions of the image that is dominated by higher frequencies. These returns are processed into an image with higher resolution in the shallower parts, and lower resolution with adequate SNR in the deeper parts.

### CROSS REFERENCE TO RELATED APPLICATIONS

This application claims the benefit of U.S. provisional application 60/867,922, filed Nov. 30, 2006, which is incorporated herein by reference.

### BACKGROUND

In medical ultrasound imaging, using higher frequencies provides better resolution. (As used herein, frequency refers to the center frequency of the signal that is used to drive the transducer.) However, since attenuation is directly proportional to frequency, increasing the frequency also cuts the depth of penetration. For example, if using a 4 MHz signal to capture an image provides a depth of penetration is 10 cm, increasing the frequency to 8 MHz will cut the depth of penetration to about 5 cm. In other words, there is a trade off between resolution and penetration: operating at lower frequencies provides more penetration and less resolution; and operating at higher frequencies provides more resolution and less penetration.

One prior art solution is to use pulses at two different frequencies for each line in the image, then combine the return echo from those pulses into a single line of the image. More specifically, in shallower portions, where there is plenty of signal power, the return from the higher frequency pulse is used to provide higher resolution. But beyond a certain point, where the signal to noise ratio (SNR) of the higher frequency return is too low to provide a good image, the return from the lower frequency pulse is used. Using this two-pulse-per-line approach however, requires twice as many pulses to obtain each ultrasound image frame (i.e., two pulses for each line in the frame instead of the more standard single pulse per line). This increases the time it takes to capture each frame of the image, increases the total ultrasound energy that is transmitted into the patient to capture the images, and increases the overall complexity of the system.

### SUMMARY

When an ultrasound transducer is driven by a signal that contains a relatively wide range of frequencies, the frequency-dependent attenuation characteristics of the subject being imaged can be relied on to simultaneously provide, using only a single pulse per line of the image, (a) a return from the deeper portions of the image that is dominated by lower frequencies and (b) a return from the shallower portions of the image that is dominated by higher frequencies. These returns are processed into an image with higher resolution in the shallower parts, and lower resolution (yet still with an adequate SNR) in the deeper parts.

### BRIEF DESCRIPTION OF THE DRAWINGS

FIG. 1 is a schematic representation of a two-cycle sinusoidal pulse that is used to drive an ultrasound transducer.

FIG. 2 is a frequency spectrum for the pulse depicted in FIG. 1.

FIG. 3 is a set of frequency response curves that show what the return signal would look like for six different depths, when the pulse depicted in FIG. 1 is used to drive the ultrasound transducer.

### DETAILED DESCRIPTION OF THE PREFERRED EMBODIMENTS

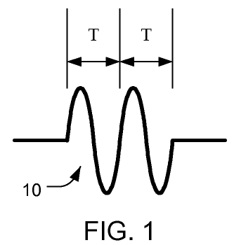

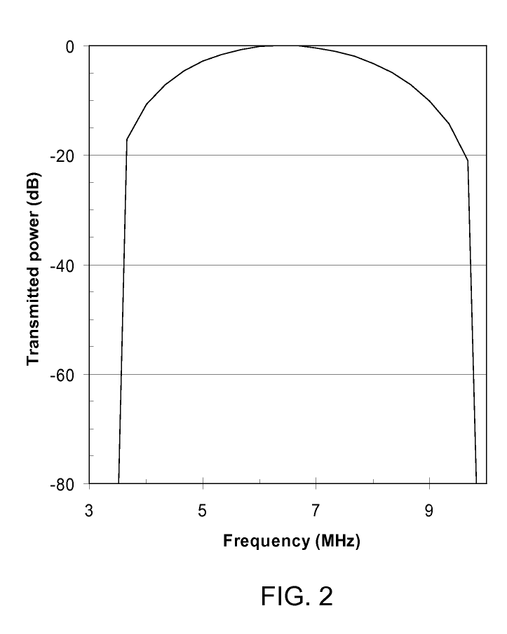

Since shorter durations in the time domain correspond to wider spectra in the frequency domain, one way to obtain the desired broadband signal is to use a short pulse. For example, if the transducer is driven by a pulse 10 that consists of two consecutive sinusoidal waves, as depicted in FIG. 1, the frequency domain equivalent will be relatively broad, as depicted in FIG. 2. For a quantitative example, if the underlying sinusoidal frequency of the signal depicted in FIG. 1 is 6.667 MHz (i.e., the period T of each full-cycle wave is 0.15 μS), the bandwidth measured from the −6 dB point (i.e., the half-power point) on the low end to the −6 dB point on the high end will be approx 4.4 to 8.4 MHz, i.e., a 4 MHz band centered around 6.4 MHz. The range 4.4 to 7 MHz is especially important (as compared with 7 to 8.4 MHz) because the higher frequencies are attenuated more with increasing depth at a rate approx 1 dB/(cm MHz) in tissue. In alternative embodiments, any function with a bandwidth greater than 2 MHz should suffice, but a bandwidth greater than 3 MHz is preferred.

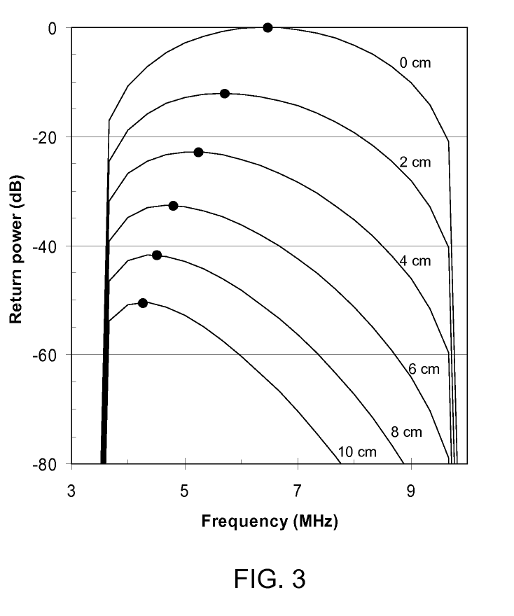

FIG. 3 is a graph that depicts the return amplitude as a function of frequency (calculated) when a hypothetical transducer with a transfer function that is totally flat between 3.33 and 9.67 MHz is driven by the two-cycle 6.67 MHz sinusoidal pulse 10 discussed above in connection with FIG. 1, for each of six different depths ranging from 0 to 10 cm. For each curve in FIG. 3, the spectral peak is indicated by a solid circle. Examination of the graph and the underlying data used to create it reveals that the return amplitude peaks at 6.44 MHz for a depth of 0 cm, at 5.74 MHz for a depth of 2 cm, and at 4.36 cm for a depth of 10 cm. It is therefore apparent that when the transducer is driven with a broadband signal, the returns originating from deeper portions of the image are centered at lower frequencies than the returns that originated from shallower portions of the image.

Similar results can be obtained with real (i.e., non-hypothetical) transducers as well, and examination of the curves in FIG. 3 for the hypothetical transducer provide insight as to what the characteristics of such real transducers should be. For example, in cases where the main region of interest is between 2 and 10 cm, a suitable center frequency for a real transducer would be midway between the return amplitude peaks at those depths (i.e., midway between 5.74 and 4.36 MHz, which comes to 5.05 MHz).

It is notable that the design point for the center frequency of the transducer, 5.05 MHz, is significantly lower than the 6.67 MHz frequency (which corresponds to a 0.15 μS period) of the sinusoidal pulse 10 used to excite the transducer. Selecting a transducer with a center frequency that is significantly lower than the frequency of the driving signal works because the attenuation of the higher frequency components is greater than for the lower frequency components, which shifts the average frequency of the received return down towards the lower frequencies. To take advantage of this shift, the frequency of the driving signal should preferably be at least 10% higher than the center frequency of the transducer, and more preferably at least 20% higher than the center frequency of the transducer.

The curves for the hypothetical transducer in FIG. 3 also suggest additional design considerations for the real transducer. For example, since the 2 cm curve peaks at 5.74 MHz and the 10 cm curve peaks at 4.36 MHz, the transducer may be optimized in some embodiments to operate at those frequencies (e.g., by having a relatively flat frequency response between 4.0 and 6.1 MHz, with a 6 dB bandwidth bounded on the low end at about 3.67 MHz and on the upper end at about 7.0 MHz). Alternatively, since the curves in FIG. 1 are not very steep near their peaks, the maximum response of the transducer may be shifted a little towards the higher frequencies to provide a corresponding increase in resolution at the expense of penetration (if such a trade off is desired by the system designer). This may be accomplished, for example, by using a transducer with a relatively flat frequency response between 5.0 and 7.0 MHz (as opposed to between 4.0 and 6.1 MHz).

This combination of transducer characteristics and driving waveform characteristics advantageously provides superior resolution for the shallower portions of the image (because the returns from those depths dominated by are higher frequency components), and maintains penetration depth for deeper portions of the image, albeit with lower resolution (because the returns from those depths are dominated by lower frequency components). Notably, both these benefits are obtained simultaneously from a single transmit pulse, without the added complexity inherent in actually transmitting two pulses at different frequencies, then receiving the returns from those two pulses, and then combining those two returns into a single image. This arrangement also reduces the amount of ultrasound energy that is transmitted into the patient (and the thermal benefits associated therewith) as compared to the prior art approach of using more than one pulse for each scan line. It also avoids image artifacts that can be introduced by motion of the subject that might occur between the high frequency pulse and the low frequency pulse, or by the algorithms that assemble the output image from the two raw ultrasound images.

If desired, the return signals from all depths may be processed using the same signal processing algorithm. Alternatively, processing of different regions of the image may be optimized based on a priori knowledge of the expected center frequency contained in each to the different regions. For example, the image may be divided into a few depth bands (e.g., a first band between 0 and 4 cm and a second band beyond 4 cm), and different signal processing parameters may be used in each of those regions (e.g., a first set of filter coefficients that is optimized for higher frequency signals may be used for the first depth band, and a second set of filter coefficients that is optimized for lower frequency signals may be used for the second depth band). As yet another alternative, the change in processing may be varied continuously as a function of depth instead of in discrete steps (e.g., by selecting filter coefficients for each pixel in the image as a function of that pixel's depth).

In alternative embodiments, waveforms other than the two consecutive sinusoidal waves depicted in FIG. 1 may also be used to drive the transducer. For example, a pulse consisting of a different number of sinusoidal waves may be used, as long as the number is small enough (e.g., 1 or 3) to have a frequency spectra that is sufficiently wide. However, the inventor has empirically determined that a two wave pulse provides better results than either a single wave pulse or a three wave pulse. Other wave shapes may also be used to drive the transducer, such as square waves, triangle waves, etc. Since those shaped inherently contain higher frequency components, the optimum number of pulses may differ as compared to when sinusoids are used. Optionally, an envelope may be imposed on the desired wave shape to impact the spectral characteristics of the wave.

While the above-described embodiments work well with existing ultrasound transducers, it is expected that customizing the bandwidth and rolloff characteristics of the ultrasound transducer to take advantage of the effects desired herein should further improve performance. For example, instead of designing the ultrasound transducer to have symmetrical rolloff characteristics (which is a common design goal for conventional ultrasound transducers), performance can be improved if is the ultrasound transducer has a steeper rolloff above the center frequency than below the center frequency. The transducer should preferably be relatively flat from about 0.65 times the nominal center frequency f to about 1.1 times f, that is over the rage 0.65 f to 1. 1 f, and roll off slowly (e.g., 6-9 dB/MHz) for 1 MHz above and below that range.

Of course, persons skilled in the relevant arts will recognize that the scope of the invention in not limited by the numeric examples provided herein (e.g., for center frequencies, rolloff rates, etc.) and that they may be adjusted based on the desired design goals of the particular system that is being implemented.

I claim:

1. A method of obtaining an ultrasound image of a region of interest, the method comprising the steps of:
driving an ultrasound transducer with a broadband signal;
receiving, using the ultrasound transducer, a portion of the broadband signal that has been reflected from the region of interest;
processing portions of the received signal that correspond to a deep section of the region of interest assuming that the center frequency is f₁; and
processing portions of the received signal that correspond to a shallow section of the region of interest assuming that the center frequency is f₂, wherein f₂ is higher than f₁.

2. The method of claim 1, wherein portions of the received signal that correspond to the deep section of the region of interest are processed by a filter optimized for detecting f₁, and portions of the received signal that correspond to the shallow section of the region of interest are processed by a filter optimized for detecting f₂.

3. The method of claim 1, further comprising the step of processing portions of the received signal that correspond to an intermediate depth section of the region of interest assuming that the center frequency is between f₁ and f₂.

4. The method of claim 1, wherein, for each line in an image, the broadband signal comprises at least one of (a) a single full-wave sinusoidal pulse (b) two contiguous full-wave sinusoidal pulses and (c) a square wave.

5. The method of claim 1, wherein, for each line in an image, the broadband signal consists of two contiguous full-wave sinusoidal pulses.

6. The method of claim 1, wherein the broadband signal has a bandwidth of at least 3 MHz, measured from the minus 6 dB point on the low frequency side to the minus 6 dB point on the high frequency side.

7. The method of claim 1, wherein the broadband signal has a bandwidth of at least 2 MHz, measured from the minus 6 dB point on the low frequency side to the minus 6 dB point on the high frequency side.

8. The method of claim 1, wherein f₂ is at least 20% higher than f₁.

9. A method of obtaining an ultrasound image of a region of interest, the method comprising the steps of:
driving an ultrasound transducer having a nominal operating frequency f_{N} with a broadband signal having a center frequency that is at least 10% higher than f_{N}, so that the ultrasound transducer transmits ultrasound energy into the region of interest;
relying on frequency-dependent attenuation characteristics of the region of interest to present return signals to the ultrasound transducer in which (a) portions of the return signals that correspond to deeper parts of the region of interest are dominated by lower frequencies and (b) portions of the return signals that correspond to shallower parts of the region of interest are dominated by higher frequencies; and
receiving the return signals using the ultrasound transducer.

10. The method of claim 9, wherein the center frequency of the higher frequencies is at least 20% higher than the center frequency of the lower frequencies, and f_{N} is at least 10% higher than the center frequency of the higher frequencies.

11. The method of claim 9, further comprising the step of processing the return signals received in the receiving step into an image.

12. The method of claim 9, wherein, for each line in an image, the broadband signal comprises at least one of (a) a single full-wave sinusoidal pulse (b) two contiguous full-wave sinusoidal pulses and (c) a square wave.

13. The method of claim 9, wherein, for each line in an image, the broadband signal consists of two contiguous full-wave sinusoidal pulses.

14. The method of claim 9, wherein the broadband signal has a bandwidth of at least 3 MHz, measured from the minus 6 dB point on the low frequency side to the minus 6 dB point on the high frequency side.

15. The method of claim 9, wherein the broadband signal has a bandwidth of at least 2 MHz, measured from the minus 6 dB point on the low frequency side to the minus 6 dB point on the high frequency side.

16. The method of claim 9, wherein the broadband signal has a center frequency that is at least 20% higher than f_{N}.

17. The method of claim 9, wherein the broadband signal has a center frequency of about 6⅔ MHz, and f_{N} is about 5 MHz.

18. An ultrasound imaging apparatus comprising:
an ultrasound transducer having a nominal operating frequency f_{N};
a transmitter operatively connected to the ultrasound transducer, wherein the transmitter drives the ultrasound transducer with a broadband signal with a center frequency that is at least 10% higher than f_{N};
a receiver operatively connected to the ultrasound transducer adapted to receive a return signal corresponding to energy reflected off of matter in a region of interest, in which (a) portions of the return signals that correspond to deeper parts of the region of interest are dominated by lower frequencies and (b) portions of the return signals that correspond to shallower parts of the region of interest are dominated by higher frequencies; and
a processor configured to process the portions of the return signals that correspond to deeper parts of the region of interest and the portions of the return signals that correspond to shallower parts of the region of interest into an image.

19. The apparatus of claim 18, wherein the center frequency of the higher frequencies is at least 20% higher than the center frequency of the lower frequencies, and f_{N} is at least 10% higher than the center frequency of the higher frequencies.

20. The apparatus of claim 18, wherein, for each line of the image, the broadband signal comprises at least one of (a) a single full-wave sinusoidal pulse (b) two contiguous full-wave sinusoidal pulses and (c) a square wave.

21. The apparatus of claim 18, wherein, for each line of the image, the broadband signal consists of two contiguous full-wave sinusoidal pulses.

22. The apparatus of claim 18, wherein the broadband signal has a bandwidth of at least 3 MHz, measured from the minus 6 dB point on the low frequency side to the minus 6 dB point on the high frequency side.

23. The apparatus of claim 18, wherein the broadband signal has a bandwidth of at least 2 MHz, measured from the minus 6 dB point on the low frequency side to the minus 6 dB point on the high frequency side.

24. The apparatus of claim 18, wherein the broadband signal has a center frequency that is at least 20% higher than f_{N}.

25. The apparatus of claim 18, wherein the broadband signal has a center frequency of about 6⅔ MHz, and f_{N} is about 5 MHz.
